(19) United States
(12) Patent Application Publication
(10) Pub. No.: US 2023/0404407 A1
(43) Pub. Date: Dec. 21, 2023

(54) THERMOACOUSTIC AND ULTRASOUND TOMOGRAPHY

(71) Applicant: California Institute of Technology, Pasadena, CA (US)

(72) Inventors: David C. Garrett, Glendale, CA (US); Jinhua Xu, Pasadena, CA (US); Geng Ku, South Pasadena, CA (US); Lihong Wang, Arcadia, CA (US)

(21) Appl. No.: 18/336,863

(22) Filed: Jun. 16, 2023

Related U.S. Application Data
(60) Provisional application No. 63/352,857, filed on Jun. 16, 2022.

(51) Int. Cl. A61B 5/00; A61B 8/00

(52) U.S. Cl. CPC A61B 5/0095; A61B 8/4494; A61B 8/4416; A61B 8/4477

(57) ABSTRACT

Certain aspects pertain to systems, apparatus, and methods for one or both of thermoacoustic and ultrasound tomography.

### CROSS-REFERENCES TO RELATED APPLICATIONS

This application claims priority to and the benefit of U.S. Provisional Patent Application No. 63/352,857, titled “SYSTEM FOR HUMAN-SCALE THERMOACOUSTIC AND ULTRASOUND TECHNOLOGY” and filed on Jun. 16, 2022, which is hereby incorporated by reference in its entirety and for all purposes.

### FEDERALLY SPONSORED RESEARCH OR DEVELOPMENT

This invention was made with government support under Grant No. CA220436 awarded by the National Institute of Health. The government has certain rights in the invention.

### FIELD

Certain aspects relate generally to the field of medical imaging, and more specifically, to thermoacoustic and ultrasound tomography.

### BACKGROUND

Thermoacoustic tomography (TAT) is a medical imaging technique that combines rich radio frequency absorption contrast and fine acoustic resolution. Modulated radio frequency signals are irradiated into tissues, leading to absorption-specific thermoelastic expansion released as thermoacoustic waves. Acoustic signals are detected outside the tissue and are used for image reconstruction of the absorption profile. The resulting images have contrast relating to water and ionic content and provide mm-scale resolution of biological structures within the body.

Ultrasound tomography (UST) is a medical imaging modality which involves transmitting ultrasonic signals into tissues and recording the signals transmitted, reflected and/or scattered by the tissue. The recorded signals are used to recover various acoustic parameters such as the speed of sound, acoustic attenuation, and acoustic impedance variations inside the tissue. Other physiological parameters such as fluid flow can be recovered.

Conventional TAT systems have been only suitable for imaging extremities such as the breast or limbs. TAT was first demonstrated for human imaging with breast imaging using 25 kW pulses of 433 MHz signals with 0.5 us pulse width and 500 Hz repetition rate as discussed in R. A. Kruger, W. L. Kiser, D. R. Reinecke, G. A. Kruger, and R. L. Eisenhart, “Thermoacoustic computed tomography of the breast at 434 MHz,” in 1999 IEEE MTT-S International Microwave Symposium Digest (Cat. No. 99CH36282), vol. 2, pp. 591-594 (1999) and R. A. Kruger et al., “Thermoacoustic CT: imaging principles,” in Biomedical Optoacoustics, vol. 3916, pp. 150-159 (May 2000). Imaging required 9.5 minutes, averaging 1024 times at 256 detector angles. Tissues were irradiated either using a single or eight waveguide antennas, with deionized water coupled around the breast. An example of imaging using a single waveguide antenna can be found in R. A. Kruger, W. L. Kiser, D. R. Reinecke, G. A. Kruger, and R. L. Eisenhart, “Thermoacoustic computed tomography of the breast at 434 MHz,” in 1999 IEEE MTT-S International Microwave Symposium Digest (Cat. No. 99CH36282), vol. 2, pp. 591-594 (1999). An example of imaging using eight waveguide antennas can be found in R. A. Kruger, K. Stantz, and W. L. Kiser, Jr., “Thermoacoustic CT of the breast,” San Diego, CA, pp. 521-525 (May 2002). Breast imaging was later performed on subjects using a 3 GHz source with 70 kW peak power and 0.5 μs pulsewidth as discussed in L. Wu et al., “A handheld microwave thermoacoustic imaging system with an impedance matching microwave-sono probe for breast tumor screening,” IEEE Trans. Med. Imaging, 202. Using a similar system, human forearm imaging was also demonstrated in Z. Zheng, L. Huang, and H. Jiang, “Label-free thermoacoustic imaging of human blood vessels in vivo,” Appl. Phys. Lett., vol. 113, no. 25, p. 253702 (2018). Existing UST systems have also been developed primarily for imaging extremity regions of soft tissue such as the breast as discussed in N. Duric et al., “Development of ultrasound tomography for breast imaging: Technical assessment,” Med. Phys., vol. 32, no. 5, pp. 1375-1386 (2005) and C. Li, N. Duric, P. Littrup, and L. Huang, “In vivo Breast Sound-Speed Imaging with Ultrasound Tomography,” Ultrasound Med. Biol., vol. 35, no. 10, pp. 1615-1628, (October 2009). These conventional TAT and UST systems are primarily limited to accessing the extremities.

### SUMMARY

Certain aspects relate generally to the field of medical imaging, and more specifically, to thermoacoustic and ultrasound tomography of, e.g., human tissues.

According to one aspect, a TAT/UST system includes a radio frequency (RF) source, radio frequency antennas, ultrasonic transmitter(s), and a plurality of ultrasonic sensors that may be used for human-scale TAT/UST imaging.

Certain aspects pertain to TAT/UST techniques that recover radio frequency absorption and ultrasonic contrast of regions deep in the body, which may enable the diagnosis and evaluation of a plurality of health conditions.

Certain aspects pertain to imaging systems having one or more dielectric-loaded radio frequency antenna apparatus, each dielectric-loaded radio frequency antenna apparatus comprising a radio frequency antenna for transmitting radio frequency pulses and an internal volume at least partially filled with a dielectric material and an ultrasonic sensor array configured to detect acoustic waves.

Certain aspects pertain to an ultrasonic sensor array apparatus having a plurality of ultrasonic transducers, a plurality of integrated pre-amplifiers in one-to-one correspondence with the ultrasonic transducers, and an ultrasonic transmitter configured to transmit ultrasound pulses.

Certain aspects pertain to a dielectric-loaded radio frequency antenna apparatus having an internal volume at least partially filled with a dielectric material, a radio frequency antenna, and a gas-filled spacer between the internal volume and a region external to the dielectric-loaded radio frequency antenna.

Certain aspects pertain to thermoacoustic and ultrasound tomography imaging methods that delay triggering of radio frequency and ultrasonic pulses to interleave thermoacoustic signals with ultrasonic signals detected by an ultrasonic sensor array, reconstruct thermoacoustic image data and ultrasonic image data, and co-register the thermoacoustic image data and the ultrasonic image data.

These and other features are described in more detail below with reference to the associated drawings.

### BRIEF DESCRIPTION OF THE DRAWINGS

The present invention is illustrated by way of example and not limitation in the figures of the accompanying drawings in which like references indicate similar elements.

FIG. 1 is block diagram of components of a TAT/UST system, according to embodiments.

FIG. 2A is an isometric drawing of components of a TAT/UST system with a plurality of radio frequency (RF) antennas, according to embodiments.

FIG. 2B depicts a cross-sectional drawing of the TAT/UST system illustrated in in FIG. 2A.

FIG. 3A is an isometric drawing depicting components of the TAT/UST system with a plurality of radio frequency antennas of FIG. 2A, according to embodiments.

FIG. 3B is a front view drawing of the components of the TAT/UST system in FIG. 3A.

FIG. 3C is a cross-sectional drawing of the components of the TAT/UST system in FIG. 3A.

FIG. 4A is an isometric drawing of components of a TAT/UST system with a single radio frequency antenna, according to embodiments.

FIG. 4B depicts a cross-sectional drawing of the TAT/UST system illustrated in in FIG. 4A.

FIG. 5A is an isometric drawing depicting components of the TAT/UST system with a single radio frequency antenna of FIG. 4A, according to embodiments.

FIG. 5B is a front view drawing of the components of the TAT/UST system in FIG. 5A.

FIG. 5C is a cross-sectional drawing of the components of the TAT/UST system in FIG. 5A.

FIG. 6A is a drawing of components of an ultrasonic sensor array apparatus, according to an embodiment.

FIG. 6B is an isometric drawing of components of the ultrasonic sensor array apparatus in FIG. 6A.

FIG. 7 is a cross-sectional drawing of components of an ultrasonic sensor array of an ultrasonic sensor array apparatus, according to an embodiment.

FIG. 8A is an isometric drawing of components of a TAT/UST system, according to embodiments.

FIG. 8B is a side view drawing of components of the TAT/UST system in FIG. 8A.

FIG. 8C is a drawing of a cross-section of the ultrasonic sensor array apparatus of the TAT/UST system in FIG. 8A.

FIG. 9 depicts a block diagram depicting control of radio frequency, ultrasonic, and digital components of a TAT/UST system, according to embodiments.

FIG. 10 is a timing diagram for triggering radio frequency and ultrasonic emissions in a TAT/UST system, according to embodiments.

FIG. 11 depicts a flowchart of a TAT/UST method, according to embodiments.

FIG. 12 is an illustration of a TAT/UST system with RF antennas configured to irradiate with microwave pulses, according to an embodiment.

FIG. 13A is an image of a simulated SAR distribution into tissue of an abdomen of a human subject in the axial (left) plane using a TAT/UST system, in accordance with an implementation.

FIG. 13B is an image of a simulated SAR distribution into tissue of an abdomen of a human subject in the sagittal (right) plane using a TAT/UST system, in accordance with an implementation.

The figures and components therein may not be drawn to scale.

### DETAILED DESCRIPTION

Various embodiments and aspects of the inventions will be described with reference to details discussed below, and the accompanying drawings will illustrate the various embodiments. The following description and drawings are illustrative of the invention and are not to be construed as limiting the invention. Numerous specific details are described to provide a thorough understanding of various embodiments of the present invention. However, in certain instances, well-known or conventional details are not described to provide a concise discussion of embodiments of the present inventions.

Reference in the specification to “one embodiment” or “an embodiment” or “another embodiment” means that a particular feature, structure, or characteristic described in conjunction with the embodiment can be included in at least one embodiment. The appearances of the phrase “in one embodiment” in various places in the specification do not necessarily all refer to the same embodiment.

### I. Introduction

Certain aspects pertain to systems, apparatus, and methods for one or both of thermoacoustic and ultrasound tomography (TAT/UST). TAT/UST techniques may be used to image extremities such as breasts or limbs, and also other regions of the body such as the abdomen, chest, or head. TAT/UST techniques can be used for whole body imaging. According to one aspect, TAT/UST techniques may co-register thermoacoustic and ultrasound image data, providing clinically useful anatomical and physiological information. For example, TAT/UST techniques may co-register contrast from thermoacoustic image data on a structural background from ultrasound image data. According to certain various aspects, TAT/UST techniques may be employed for reflection-mode ultrasound imaging, transmission-mode quantitative ultrasound imaging, and thermoacoustic tomography.

Various aspects pertain to an imaging system including one or more dielectric-loaded radio frequency antenna apparatus with an internal volume at least partially filled with a dielectric material. The dielectric material may have permittivity that is the same or nearly the same as the specimen imaged during operation to aid in transmitting radio frequency pulses from the one or more dielectric-loaded radio frequency antenna apparatus into the specimen being imaged. In some cases, the one or more dielectric-loaded radio frequency antenna apparatus may also include a gas-filled (e.g., air-filled) spacer configured to block acoustic waves from propagating into the specimen being imaged. The imaging system also includes an ultrasonic sensor array configured to detect acoustic waves from, e.g., an acoustic medium in a tank in which the specimen being imaged is at least partially submerged. Alternatively or additionally, the imaging system includes an ultrasonic transmitting transducer for transmitting ultrasound pulses. The imaging system may also include a delay generator that is configured to introduce delays between the ultrasound pulses and the radio frequency pulses to interleave the thermoacoustic signals (from thermoelastic expansion) and reflected/transmitted ultrasonic signals being recorded in a single session. In one example, the ultrasonic transmitting transducer is configured to move around the specimen being imaged while the thermoacoustic signals and ultrasonic signals are recorded.

Certain aspects pertain to an ultrasonic array apparatus including one or more dielectric-loaded radio frequency antenna apparatus with an internal volume at least partially filled with a dielectric material. The dielectric material may match the impedance of a specimen imaged during operation to aid in transmitting radio frequency pulses from the one or more dielectric-loaded radio frequency antenna apparatus into the specimen being imaged. In some cases, the one or more dielectric-loaded radio frequency antenna apparatus may also include a gas-filled (e.g., air-filled) spacer configured to block acoustic waves from propagating into the specimen being imaged. In some cases, the ultrasonic array apparatus includes a motor configured to move the ultrasonic transmitting transducer along a line, an arc, or a circle. In one example, the ultrasonic array apparatus also includes a rotational shaft and a gear rotatably coupled to the rotational shaft wherein the ultrasonic transmitting transducer is coupled to the gear. In this example, the motor rotates the rotation shaft, which rotates the gear, moving the ultrasonic transmitting transducer.

Certain aspects pertain to a dielectric-loaded radio frequency antenna apparatus with an internal volume at least partially filled with a dielectric material, a radio frequency antenna, and a gas-filled spacer between the internal volume and a region external to the dielectric-loaded radio frequency antenna. The dielectric material may match the permittivity of a specimen imaged during operation to aid in transmitting radio frequency pulses from the one or more dielectric-loaded radio frequency antenna apparatus into the specimen being imaged.

In one embodiment, a TAT/UST system includes a radio frequency source, one or more radio frequency antennas, one or more ultrasonic transmitters, and a plurality of ultrasonic sensors (transducers) that can be used for human-scale imaging.

According to various aspects, TAT/UST imaging systems and methods may be used to recover radio frequency absorption and ultrasonic contrast of regions deep in the body. The recovered information can enable the diagnosis and evaluation of a plurality of health conditions.

### II. TAT/UST Systems, Radio Frequency Antenna Apparatus, and Ultrasonic Sensor Array Apparatus

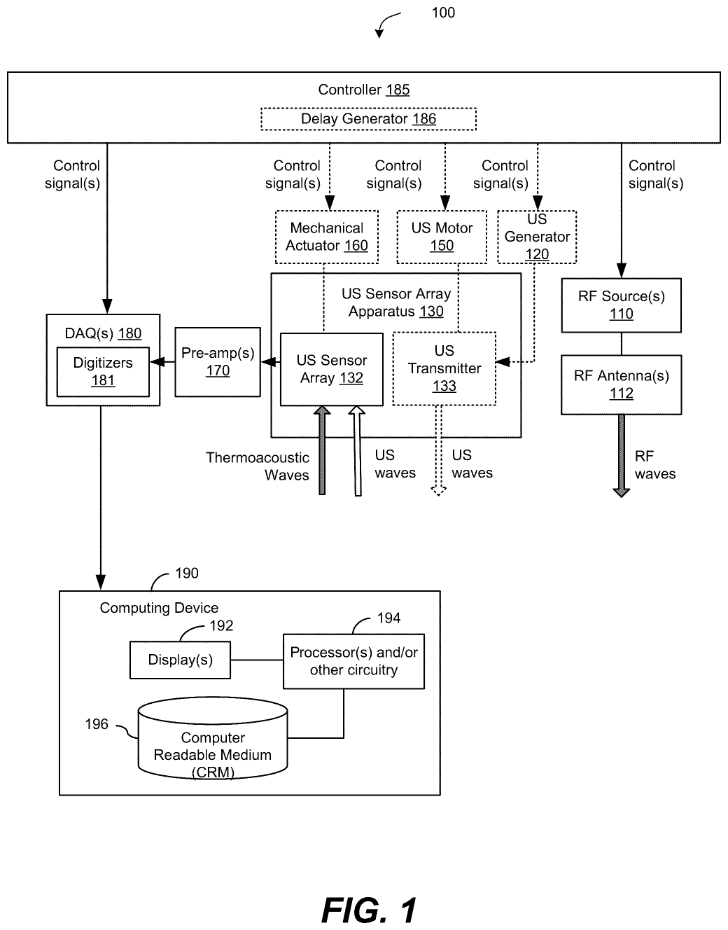

FIG. 1 is a block diagram of components of a TAT/UST system 100, according to various embodiments. TAT/UST system 100 includes one or more radio frequency (RF) sources 110 (e.g., microwave source) and one or more radio frequency antennas 112 in communication with RF source(s) 110 to receive RF energy. During operation, radio frequency antenna(s) 112 may irradiate a specimen being imaged with ultrasonic pulses. The specimen may be submerged in an acoustic medium (e.g., water solution) in e.g., a tank. The acoustic medium may be employed to couple acoustic signals in/out of the specimen and/or attenuate stray radio frequency signals. Optionally (denoted by dashed line), TAT/UST system 100 includes an ultrasonic generator 120 for providing ultrasonic signals that are propagated to the specimen being imaged. TAT/UST system 100 also includes an ultrasonic sensor array apparatus 130 that includes an ultrasonic sensor array 132 with a plurality of ultrasonic transducer elements that can detect thermoacoustic waves and ultrasonic waves. The ultrasonic transducer elements are arranged, for example, as a full-ring (circular) array, a linear array, a hemispherical array, an arc-shaped array, a two-dimensional rectangular array, etc. Optionally (denoted by dashed line), ultrasonic sensor array apparatus 130 also includes at least one ultrasonic transmitting transducer 133 (also sometimes referred to herein as a “transmitter”) in communication with ultrasonic generator 120 to receive ultrasonic energy. The optional ultrasonic transmitting transducer 133 may be employed to irradiate specimen being imaged with ultrasonic waves during operation. Optionally (denoted by dashed line), TAT/UST system 100 includes an ultrasonic motor 150 that is operably coupled to the at least one ultrasonic transmitting transducer 133 for movement of ultrasonic transmitting transducer 133. For example, during operation ultrasonic transmitting transducer 133 may be moved along a circumference of a ring while periodically providing signals of ultrasonic waves.

According to certain aspects, TAT/UST system 100 may operate in a thermoacoustic tomography mode, an ultrasonic tomography mode or a combined thermoacoustic and ultrasonic tomography mode in, e.g., a single recording session. During an exemplary method in the combined thermoacoustic and ultrasonic tomography mode, ultrasonic generator 120 and RF source(s) 110 are triggered to interleave the transmission of pulses of RF waves from radio frequency antenna(s) 112 with signals of ultrasonic waves from the at least one ultrasonic transmitting transducer 133. The interleaved pulses of RF and ultrasonic waves may irradiate specimen being imaged during operation. The pulses of RF waves irradiating the specimen cause absorption-specific thermoelastic expansion released as thermoacoustic waves. During operation, ultrasonic sensor array 132 is acoustically coupled with the specimen being imaged to detect acoustic waves generated by thermoelastic expansion in the specimen and to detect ultrasonic waves transmitted, reflected, and/or scattered by the specimen. For example, TAT/UST system 100 may include a tank of acoustic medium (e.g., water or acoustic gel) and the specimen (e.g., whole human body) may be at least partially submerged in the acoustic medium during image acquisition. Although a specimen is shown certain drawings for illustration purposes, it would be understood that the specimen is not necessarily a component of the TAT/UST system.

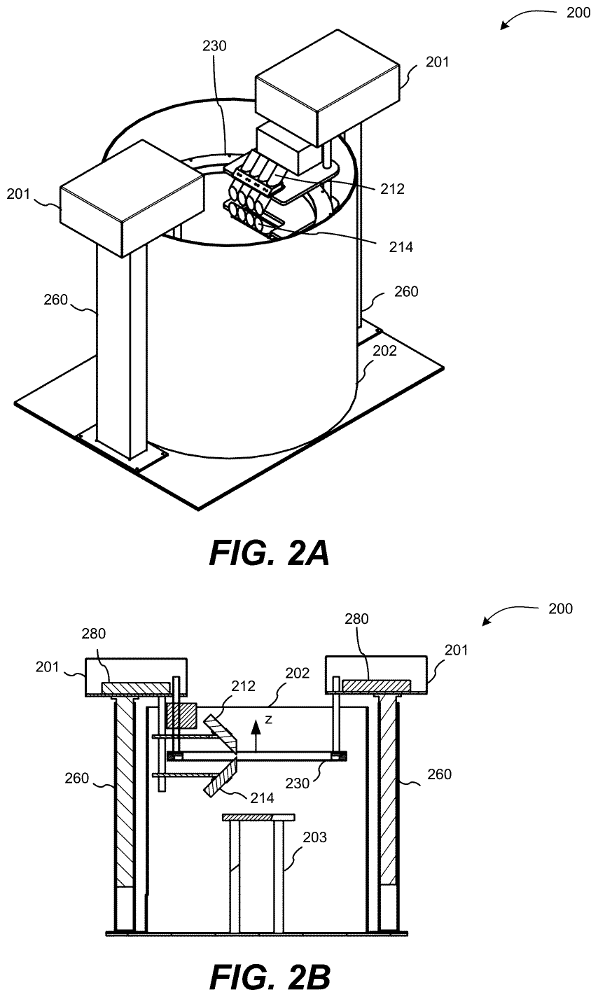

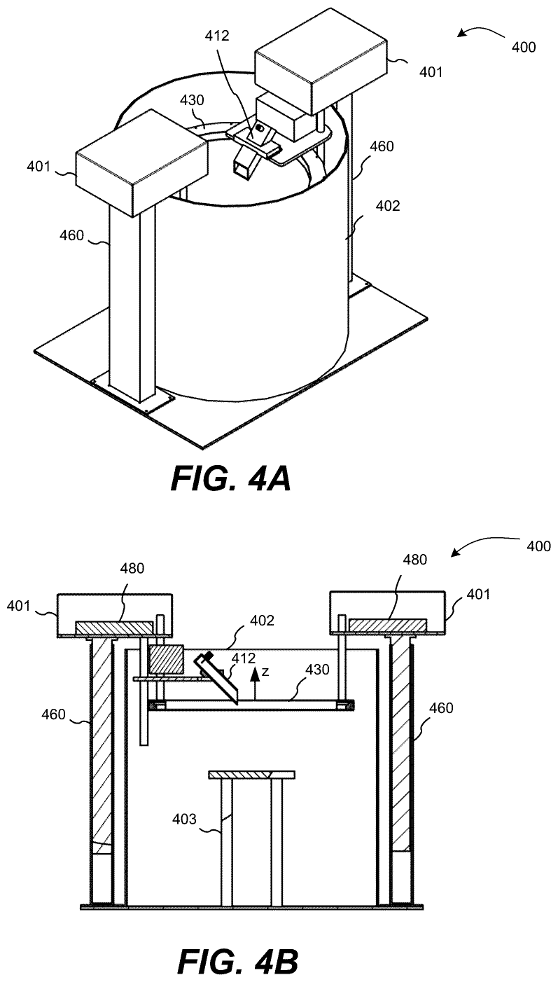

Optionally (denoted by dotted line), TAT/UST system 100 may include a mechanical actuator 160 (e.g., a linear stage such as the KR4610D linear stage made by THK America, Inc.) that may be employed for three-dimensional imaging. The mechanical actuator 160 is operably coupled to ultrasonic sensor array 132 to be able to move ultrasonic sensor array 132 to a series of elevational positions along an axis (e.g., elevational positions z₁, z₂, etc. along z-axis in FIGS. 2B and 4B, for example) and hold ultrasonic sensor array 132 at each position for a period of time during which a two-dimensional (slice) image may be acquired. Some examples of time periods that may be used include about (e.g., +−1%) 10 seconds, about 15 seconds, and about 20 seconds. According to one aspect, the time period may be in a range of about 10 seconds to about 20 seconds.

TAT/UST system 100 also includes one or more pre-amplifiers 170 for increasing amplitude (boosting) of signals received from the ultrasonic sensor array 132. TAT/UST system 100 also includes one or more data acquisition systems (DAQs) 180 with a plurality of digitizers 181 for digitizing thermoacoustic and ultrasonic signals received from one or more pre-amplifiers 170 to generate interleaved thermoacoustic and ultrasound data. Pre-amplifier(s) 170 is shown in electrical communication (e.g., directly or via other circuitry) with ultrasonic sensor array 132 to receive interleaved thermoacoustic and ultrasonic signals from the ultrasonic sensor array 132. DAQ(s) 180 is shown in electrical communication (e.g., directly or via other circuitry) with pre-amplifier(s) 170 to receive boosted thermoacoustic signals and ultrasonic signals from pre-amplifier(s) 170.

TAT/UST system 100 includes a controller 185. The controller 185 is in electrical communication with RF source(s) 110, optional ultrasonic generator 120, optional ultrasonic motor 150, mechanical actuator 160, and with DAQ(s) 180 to send control signals. Optionally, controller 185 includes a delay generator 186 that can send trigger signals to RF source(s) 110 and optional ultrasonic generator 120 to generate time delays in the transmission of pulses of RF waves and the signals of ultrasonic waves to interleave the RF pulses with the ultrasonic signals. Optionally, controller 185 may be in electrical communication with ultrasonic motor 150, mechanical actuator 160, and DAQ(s) 180 to synchronize recording of interleaved thermoacoustic and ultrasonic signals at different positions of the ultrasonic sensor array 132 and ultrasonic transmitting transducer 133. Optionally, controller 185 may also be in electronic communication with one or more pre-amplifiers 170 to send control signals to adjust amplification. Controller 185 may include one or more processors and other circuitry and computer readable media.

TAT/UST system 100 also includes a computing device 190 with one or more processors and/or other circuitry 194, an optional display 192 in electrical communication with the processor(s) 184, and a computer readable media (CRM) 186 (e.g., a non-transitory computer readable media) in electronic communication with the processor(s) or other circuitry 194. Computing device is in electronic communication with DAQ(s) 180 to receive interleaved thermoacoustic and ultrasonic data. Processor(s) and/or other circuitry 194 are in electrical communication with CRM 186 to store and/or retrieve data. The one or more processor(s) and/or other circuitry 194 are in electrical communication with optional display 192 for, e.g., displaying images. Although not shown, computing device 190 may also include a user input device for receiving data from an operator of TAT/UST system 100. The computing device 190 may be, for example, a personal computer, an embedded computer, a single board computer (e.g., Raspberry Pi or similar), a portable computation device (e.g., tablet), or any other computation device or system of devices capable of performing the functions described herein. Optionally, computing device 190 may be in communication with controller 185 to send control signals with control and synchronization data. In one aspect, the computing device 190 and controller 185 may be combined in a single apparatus.

In one aspect, the one or more processors and/or other circuitry 194 may execute instructions stored on the CRM 186 to perform one or more operations of a TAT/UST method. Processor(s) 184 and/or other circuitry 194 may execute instructions for: 1) communicating control signals to one or more components of TAT/UST system 100, 2) reconstruct thermoacoustic images from the thermoacoustic data and reconstruct ultrasound images from the ultrasonic data, and/or 3) co-register thermoacoustic image data and ultrasonic image data. The computer readable media (CRM) 186 may be, e.g., a non-transitory computer readable media. In one implementation, thermoacoustic signals and ultrasound signals detected by the ultrasonic sensor array 132 may be streamed to computing device 190 by DAQ(s) 180. After which, computing device 190 may reconstruct a sequence of thermoacoustic and ultrasound images.

The electrical communication between components of a TAT/UST system may be in wired and/or wireless form. One or more of the electrical communications between components of a TAT/UST system may be able to provide power in addition to communicate signals. During operation, digitized data may be first stored in an onboard buffer, and then transferred to the computing device of a TAT/UST system, e.g., through a universal serial bus 2.0.

In some implementations, a TAT/UST system includes one or more communication interfaces (e.g., a universal serial bus (USB) interface). Communication interfaces can be used, for example, to connect various peripherals and input/output (I/O) devices such as a wired keyboard or mouse or to connect a dongle for use in wirelessly connecting various wireless-enabled peripherals. Such additional interfaces also can include serial interfaces such as, for example, an interface to connect to a ribbon cable. It should also be appreciated that the various system components can be electrically coupled to communicate with various components over one or more of a variety of suitable interfaces and cables such as, for example, USB interfaces and cables, ribbon cables, Ethernet cables, among other suitable interfaces and cables.

In various implementations, a TAT/UST system includes one or more RF sources configured to generate pulses of RF energy and one or more RF antennas coupled to the one or more RF sources. In one example, a TAT/UST system includes one or more RF sources configured to radiate brief (e.g., less than 1 μs) microwave pulses with peak power in a range of kW. In another example, a TAT/UST system includes one or more RF sources and RF antennas configured to transmit pulses of microwaves having, for example, a frequency of about GHz. In certain implementations, an RF source may include a pulsed cavity magnetron. For example, an RF source may include a 400 kW, 1.1 GHz pulsed cavity magnetron which applies 0.2-1.0 μs pulses at fᵣₑₚ of 10-100 Hz. In implementations with multiple RE antennas, the RE source(s) may be divided into multiple channels, each channel electrically connected to one of the RF antennas. Other examples of RF sources that can be employed in other implementations include spark gap generators, solid-state amplifiers, traveling wave tube amplifiers and klystrons. In some implementations, the one or more RF antennas of a TAT/LIST system have a generally cylindrical shape. Other shapes can be used in other implementations.

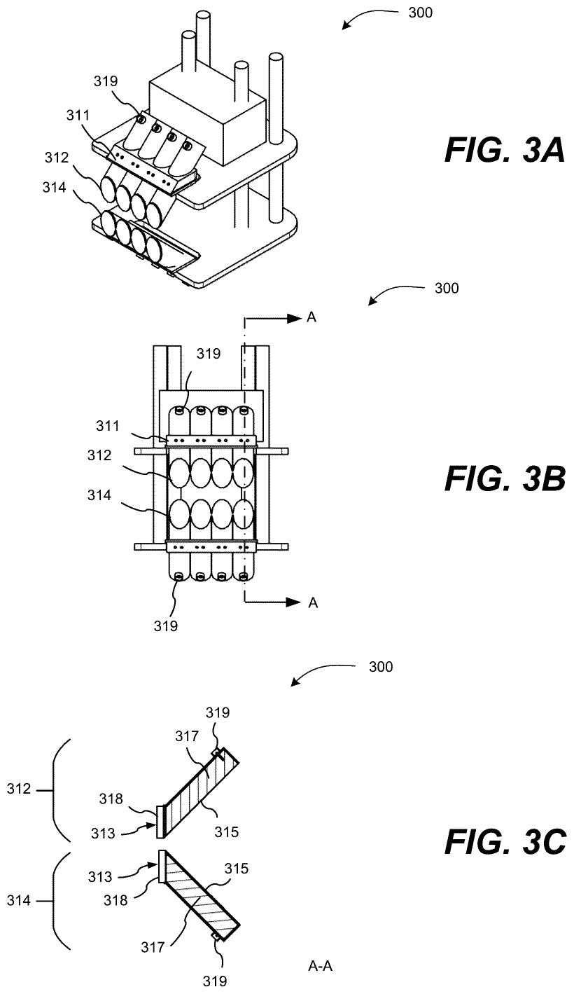

In certain implementations, a TAT/UST system includes multiple sets of similarly oriented RF antennas, for example, where each RF antenna in a set has a central axis that is parallel to the central axis of the other RF antennas in the set. For example, TAT/UST system 200 in FIGS. 2A-2B and FIGS. 3A-3C has a first set of RF antennas 212 having a central axis that is parallel to the other RF antennas 212 in the first set and a second set of RF antennas 214 having a central axis x″ that is parallel to the other RF antennas 214 in the second set.

According to various implementations, a TAT/UST system includes one or more RF antenna apparatus configured to transmit pulses of RF waves to a specimen being imaged during operation. The RF antenna apparatus generally includes an outer wall that is an RF waveguide antenna. The RF antenna apparatus includes a distal end (also sometimes referred to herein as a specimen-facing side) that may be configured to at least partially contact the specimen being imaged during operation.

In various implementations, the RF antenna apparatus is a dielectric-loaded waveguide antenna with a volume or space within the outer wall that is at least partially filled with a dielectric material that can aid in coupling radiofrequency energy into the specimen (e.g., tissue) being imaged. In some cases, the dielectric material may match (e.g., with about 20) match the permittivity of the specimen being imaged. In one example, the dielectric material has a permittivity of about 10 or about 25 to match the tissue permittivity of around 30.

In addition or alternatively, the RF antenna apparatus may have a gas-filled spacer (e.g., air-filled spacer) at, or near, the distal end. The gas-filled spacer may substantially block thermoacoustic signals that may be generated by the RF waveguide antenna from propagating to the specimen through the distal end. In one aspect, the gas-filled spacer has an air gap with a thickness of about 1 mm.

In various implementations, a TAT/UST system includes an ultrasonic sensor array apparatus having an ultrasonic sensor array with a plurality of N transducers (sometimes referred to herein as “transducer elements”) that are configured or configurable to collect acoustic signals, e.g., in parallel. Each transducer element has an aperture (e.g., a flat-rectangular aperture). The plurality of transducers in the ultrasonic sensor array have a height, a width, and a pitch between transducers. The transducer elements may be in arranged in a linear array, a full-ring (circular) array, a hemispherical array, an arc-shaped array, a two-dimensional rectangular array, etc. According to one aspect, a full-ring ultrasonic sensor array may be employed. For example, a 512-element full-ring ultrasonic sensor array may be used with transducer elements equally distributed along the circumference of a ring having a diameter and a constant inter-element spacing. The ring diameter may be at least 220 mm in one aspect, may be at least 200 mm in one aspect, or may be at least 250 mm in one aspect. In one aspect, the ring diameter is in a range of about 150 mm to about 400 mm. In one aspect, the ring diameter is about 600 mm. The inter-element spacing may be less than or equal to about 1.0 mm in one aspect, less than or equal to mm in one aspect, less than or equal to 1.5 mm in one aspect, or less than or equal to 2.0 mm in one aspect. In one aspect, the inter-element spacing is in a range of 0 mm to about 5 mm. In one aspect the inter-element spacing is about 3.7 mm.

In one aspect, the ultrasonic sensor array apparatus includes integrated pre-amplifiers. For example, the ultrasonic sensor array apparatus may include pre-amplifiers and ultrasonic transducers electrically connected to a printed circuit board (PCB) where each pre-amplifier is electrically connected to one ultrasonic transducer (integrated pre-amplifier and ultrasonic transducer pair). In an implementation with a full-ring ultrasonic sensor array having integrated pre-amplifiers, the integrated pre-amplifier and ultrasonic transducer pairs may be positioned along the circumference of the ring, which may be positioned around the specimen being imaged during operation.

In some implementations, the ultrasonic receiver transducers of the ultrasonic sensor array are unfocused transducers. In one aspect, ultrasonic receiver transducers may have an elevational (vertical) orientation of about 10 degrees or less and an in-plane angle of about 60 degrees. The ultrasonic receiver transducers may be 10 mm tall and 3 mm wide.

In one aspect, each unfocused transducer has a central frequency in a range of 0.50 MHz to 2.25 MHz and a one-way bandwidth of more than 50%. In another aspect, each unfocused transducer has a central frequency in a range of 2.25 MHz to 10 MHz and a one-way bandwidth of more than 50%.

According to various embodiments, a TAT/UST system includes one or more mechanical actuators (e.g., motorized translation stage) that can linearly scan or move the ultrasonic sensor array apparatus along an axis to one or more elevational positions. At each elevational position, the TAT/UST system may acquire a two-dimensional (slice) image. For example, the TAT/UST system 100 in FIGS. 2A and 2B include two mechanical actuators 260 and the TAT/UST system 300 in FIGS. 4A and 4B include two mechanical actuators 460. The mechanical actuator(s) may be coupled to the ultrasonic sensor array to move/scan the array in, for example, one or both directions along an axis (e.g., z-axis in FIG. 2B and z-axis in FIG. 4B). The mechanical actuator(s) may include, for example, a linear actuator, a linear ball screw assembly, a linear stage, or one or more motorized scanning stages.

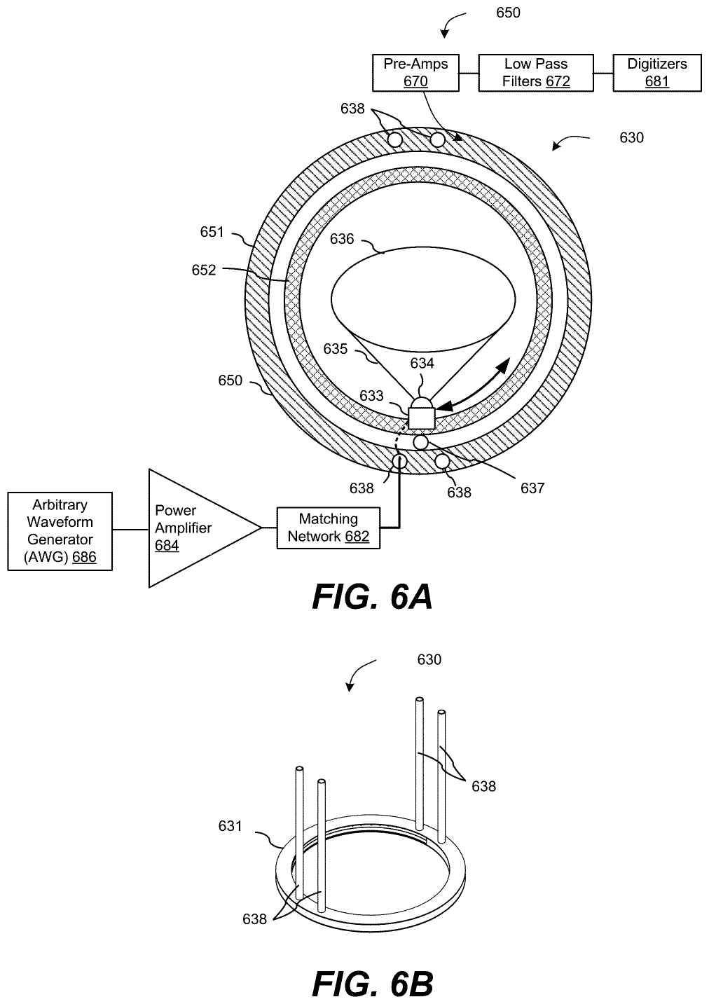

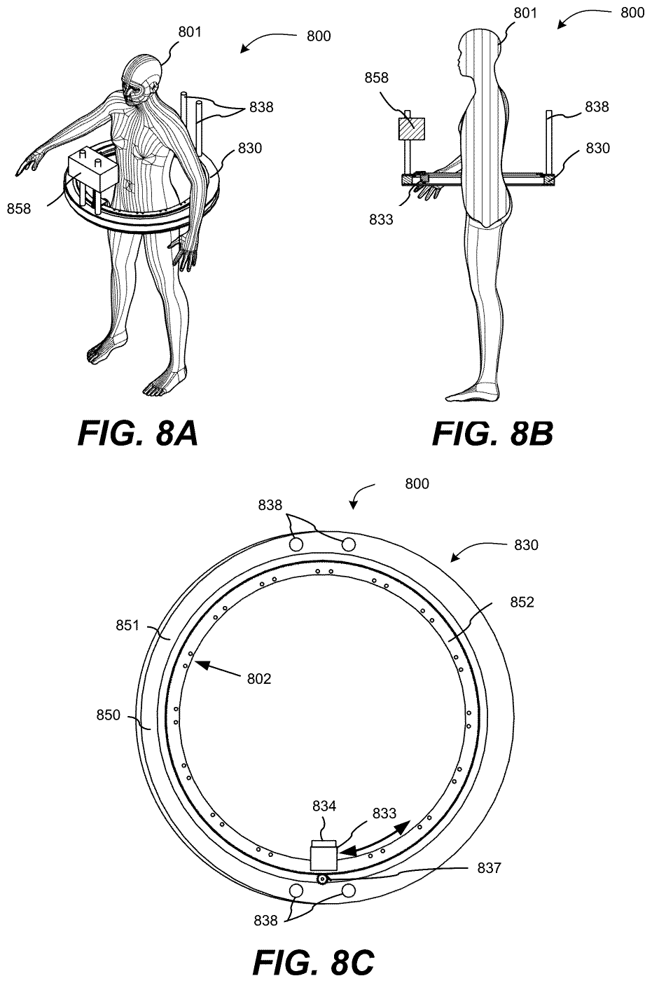

In various implementations, a TAT/UST system includes an ultrasonic transmitting transducer element for transmitting signals of ultrasonic waves to a specimen and an ultrasonic motor coupled to the ultrasonic transmitting transducer to move the ultrasonic transmitting transducer along a path during operation. For example, the ultrasonic motor may be configured to move the ultrasonic transmitting transducer around the specimen being imaged. In a ring sensor array embodiment, for example, the ring sensor array apparatus (e.g., ultrasonic sensor array apparatus 630 in FIG. 6A or ultrasonic sensor array apparatus 830 in FIG. 8C) may include a motorized rotational shaft attached to the ultrasonic motor and a mechanical gear coupled to the rotational shaft. During operation, the ultrasonic motor may be employed to rotate the motorized rotational shaft, which rotates the mechanical gear and moves (rotates) the ultrasonic transmitting transducer along a circular path around a specimen being imaged. In some cases, the ultrasonic transmitting transducer includes a diverging lens.

The one or more DAQs of a TAT/UST system record acoustic signals according to a sampling frequency. In one aspect, the sampling frequency is about 5 MHz. In another aspect, the sampling frequency is about 40 MHz. In some cases, the sampling frequency is in a range from about 4 MHz to about 100 MHz.

According to one aspect, the digitizers of the DAQ(s) and/or pre-amplifiers of a TAT/UST system are in one-to-one mapped associations with the ultrasonic transducers in the ultrasonic sensor array. These one-to-one mapped associations allow for fully parallelized data acquisition of all ultrasonic transducer channels and avoids the need for multiplexing after each pulse. With one-to-one mapped associations between pre-amplifiers and transducer elements, each transducer element in the array is in electrical communication with one dedicated pre-amplifier channel (also referred to as “preamp channel”). The one dedicated pre-amplifier channel is configured to amplify only acoustic signals detected by the one associated/mapped ultrasound transducer. These one-to-one mapped associations between the transducers and the pre-amplifier channels allow for parallelized pre-amplification of the acoustic and ultrasonic signals detected by the plurality of transducers in the ultrasound sensor array. With one-to-one mapped analog-to-digital sampling, each pre-amplifier is operatively coupled to a corresponding dedicated data channel of an analog-to-digital sampling device in a DAQ to enable parallelized analog-to-digital sampling of the plurality of pre-amplified signals. The pre-amplified signals produced by each individual preamp channel are received by a single dedicated data channel of the at least one analog-to-digital sampling devices. Any suitable number of pre-amplifier devices and/or DAQ devices may be used to provide the one-to-one mapping. For example, a TAT/UST system may include four 128-channel DAQs (e.g., SonixDAQ made by Ultrasonix Medical ULC with 40 MHz sampling rate, 12-bit dynamic range, and programmable amplification up to 51 dB) in communication with four 128-channel pre-amplifiers to provide simultaneous one-to-one mapped associations with a 512-element sensor array. The plurality of pre-amplifier channels may be directly coupled to the corresponding plurality of ultrasound transducers or may be coupled with electrical connecting cables. In one aspect, wireless communication may be employed.

Each of the one or more pre-amplifiers of a TAT/UST system may be set to a pre-amplifier gain. The pre-amplifier gain may be determined based on one or more of a minimum signal-to-noise ratio and one or more operating parameters of the data acquisition and processing system components such as analog-to-digital sampling devices (digitizers) of the DAQs, signal amplifiers, buffers, and the computing device. In one aspect, the pre-amplifier gain is in a range that is high enough to enable transmission of the photoacoustic signals with minimal signal contamination, but below a gain that may saturate the dynamic ranges of the DAQ system used to digitize the photoacoustic signals amplified by the pre-amplifier(s). In certain aspects, the gain of the plurality of pre-amplifier channels may be at least about 5 dB, at least about 7 dB, at least about 9 dB, at least about 11 dB, at least about 13 dB, at least about 15 dB, at least about 17 dB, at least about 19 dB, at least about 21 dB, at least about 23 dB, at least about 25 dB, or at least about 30 dB. In one aspect, the pre-amplifier channels are in a range of about 15-20 dB gain.

In certain implementations, a TAT/UST system includes a tank (e.g., tank 202 in FIGS. 2A and 2B or tank 402 in FIGS. 4A and 4B) that is at least partially filled with an acoustic medium such as water or a water solution. The specimen being imaged during operation such as a human subject may be located directly in the acoustic medium or in a portion of the tank that is submerged or otherwise located in the acoustic medium. The tank may be employed to couple acoustic signals in/out of the specimen and/or attenuate stray radio frequency signals.

In certain implementations, a TAT/UST system includes one or more electromagnetic shielding elements around one or electrical components to block waves generated by the electrical components from propagating to the ultrasonic sensor array. For example, the ultrasonic sensor array apparatus may include a shielding, e.g., a stainless steel shielding. The shielding may be part of a housing around the one or more electrical components.

FIG. 2A is an isometric drawing of components of a TAT/UST system 200 with a plurality of radio frequency (RF) antenna apparatus 212, 214, according to embodiments. FIG. 2B depicts a cross-sectional drawing of the TAT/UST system 200 illustrated in in FIG. 2A. TAT/UST system 200 may be capable of human-scale imaging. TAT/UST system 200 includes a tank 202 at least partially filled with an acoustic medium such as a water solution. TAT/UST system 200 also includes a seat 203 for a human specimen.

The plurality of radio frequency (RF) antenna apparatus 212, 214 includes a set of four (4) first RF antenna apparatus 212 and a set of four (4) second RF antenna apparatus 214. Each RF apparatus includes an outer wall that functions as an RF waveguide antenna for propagating RF waves. The plurality of RF antenna apparatus 212, 214 is configured to couple radio frequency energy (e.g., pulses of RF waves) into a specimen being imaged such as tissue of a human body. In other implementations, additional or fewer RF antenna apparatus may be used. Each RF antenna apparatus 212, 214 may be in communication with one or more RF sources to receive RF energy. During operation, the RF waves propagated to the specimen being images can cause absorption-specific thermoelastic expansion, released as thermoacoustic waves. In one aspect, TAT/UST system 200 also includes at least one ultrasonic transmitter for transmitting ultrasonic pulses.

TAT/UST system 200 also includes an ultrasonic sensor array apparatus 230 with an ultrasonic sensor array having a series of ultrasonic transducers that can record the resulting thermoacoustic and/or ultrasonic signals received from, e.g., the acoustic medium in tank 202. The ultrasonic transducers may be coupled in parallel (one-to-one correspondence) to integrated pre-amplifiers in the ultrasonic sensor array. TAT/UST system 200 also includes two mechanical actuators 260 coupled to the ultrasonic sensor array apparatus 230 to enable movement of ultrasonic sensor array apparatus 230 along a z-axis to one or more elevational positions.

TAT/UST system 200 also includes a shielded housing 201 that is an enclosure for housing one or more electrical components of TAT/UST system 200. Shielded housing 201 includes a shielding material that may block acoustic waves generated by the enclosed electrical components from propagating to the ultrasonic sensor array. TAT/UST system 200 also includes a plurality of digitizers 280 housed inside shielded housing 201. The digitizers 280 are coupled in parallel (one-to-one correspondence) to integrated pre-amplifiers of the ultrasonic sensor array to sample ultrasonic signals in parallel.

During operation, the plurality of RF antenna apparatus 212, 214 may be placed in contact with a specimen being imaged to couple the radio frequency energy into the specimen. For example, distal ends of one or more of the first RF antenna apparatus 212 and second RF antenna apparatus 214 may be placed in contact with skin (skin-coupled) of a human subject being imaged. RF antenna apparatus 212, 214 may be used to irradiate at least a section of tissue submerged in the acoustic medium of tank 202, for example. The generated thermoacoustic signals are coupled through the acoustic medium to the ultrasonic transducer elements of the ultrasonic sensor array. The thermoacoustic signals are digitized and recorded by the integrated digitizers and the thermoacoustic data is transmitted to a computing device. In one aspect, ultrasonic signals may also be transmitted onto the specimen tissue through the acoustic medium, and the transmitted, reflected, and/or scattered ultrasonic signals may be detected by the ultrasonic transducers. The ultrasonic signals may also be digitized and recorded by the integrated digitizers and the thermoacoustic data transmitted to the computing device.

FIG. 3A is an isometric drawing depicting components of a TAT/UST system 300 with a plurality of radio frequency (RF) antenna apparatus 312, 314, according to embodiments. FIG. 3B is a front view drawing of components of TAT/UST system 300 in FIG. 3A. FIG. 3C is a cross-sectional drawing of components of TAT/UST system 300 in FIG. 3A.

The plurality of RF antenna apparatus 312, 314 includes a set of four (4) first RF antenna apparatus 312 and a set of four (4) second RF antenna apparatus 314. The plurality of RF antenna apparatus 312, 314 is configured to couple radio frequency energy (e.g., pulses of RF waves) into a specimen being imaged. In other implementations, additional or fewer RF antenna apparatus may be used.

Each RF apparatus 312, 314 includes an outer wall 315 that functions as an RF antenna and a distal end 313. Each RF antenna apparatus 312, 314 also includes a radio frequency connector 319 attached to outer wall 315. The radio frequency connector 319 is in electrical communication with at least one RF source. The pulses of radio frequency energy are sent from the at least one RF source to the RF antennas via the radio frequency connectors 319.

In the illustrated example, each RF antenna apparatus 312, 314 includes an inner volume 317 at least partially filled with dielectric material. The dielectric material may improve coupling of radio frequency energy into a specimen being imaged. In one aspect, the dielectric material has a permittivity property that is the same, or approximately the same (e.g., within 10%), as the same permittivity as the specimen being imaged. For example, the dielectric material may have the same permittivity as a biological tissue. Each RF antenna apparatus 312, 314 also includes a gas-filled spacer 318 (e.g., an air-filled spacer) at the distal end 313. Gas-filled spacer 318 may block thermoacoustic signals generated inside the RF antenna apparatus. During operation, distal end 313 may be the side of the RF antenna apparatus 312, 314 facing the specimen to help prevent thermoacoustic signals generated inside the RF antenna apparatus 312, 314 from propagating into the specimen.

FIG. 4A is an isometric drawing of components of a TAT/UST system 400 with a single radio frequency (RF) antenna apparatus 412, according to embodiments. FIG. 4B depicts a cross-sectional drawing of the TAT/UST system 400 illustrated in in FIG. 4A. TAT/UST system 400 may be capable of human-scale imaging. TAT/UST system 400 includes a tank 402 at least partially filled with an acoustic medium such as a water solution. TAT/UST system 400 also includes a seat 403 for a human specimen.

The radio frequency (RF) antenna apparatus 412 includes an outer wall that functions as an RF waveguide antenna for propagating RF waves and a distal end. The RF antenna apparatus 412 is configured to couple radio frequency energy (e.g., pulses of RF waves) into a specimen being imaged such as tissue of a human body. The RF antenna apparatus may be in communication with one or more RF sources to receive RF energy. During operation, the RF waves propagate to the specimen being imaged, which causes absorption-specific thermoelastic expansion, released as thermoacoustic waves. In one aspect, TAT/UST system 400 also includes at least one ultrasonic transmitter for transmitting ultrasonic pulses.

TAT/UST system 400 also includes an ultrasonic sensor array apparatus 430 with an ultrasonic sensor array having a series of ultrasonic transducers that can record the resulting thermoacoustic and/or ultrasonic signals received from, e.g., the acoustic medium in tank 402. The ultrasonic transducers may be coupled in parallel (one-to-one correspondence) to integrated pre-amplifiers in the ultrasonic sensor array. TAT/UST system 400 also includes two mechanical actuators 460 coupled to the ultrasonic sensor array apparatus 430 to enable movement of ultrasonic sensor array apparatus 430 along a z-axis to one or more elevational positions.

TAT/UST system 400 also includes a shielded housing 401 that is an enclosure for housing one or more electrical components of TAT/UST system 400. Shielded housing 401 includes a shielding material that may block acoustic waves generated by the enclosed electrical components from propagating to the ultrasonic sensor array. TAT/UST system 400 also includes a plurality of digitizers 480 housed inside shielded housing 401. The digitizers 480 are coupled in parallel (one-to-one correspondence) to integrated pre-amplifiers of the ultrasonic sensor array to sample ultrasonic signals in parallel.

During operation, the RF antenna apparatus 412 may be placed in contact with a specimen being imaged to couple the radio frequency energy into the specimen. For example, the distal end of the RF antenna apparatus 412 may be placed in contact with skin (skin-coupled) of a human subject being imaged. RF antenna apparatus 412 may be used to irradiate at least a section of tissue submerged in the acoustic medium of tank 402, for example. The generated thermoacoustic signals are coupled through the acoustic medium to the ultrasonic transducer elements of the ultrasonic sensor array. The thermoacoustic signals are digitized and recorded by the integrated digitizers and the thermoacoustic data is transmitted to a computing device. In one aspect, ultrasonic signals may also be transmitted onto the specimen tissue through the acoustic medium, and the transmitted, reflected, and/or scattered ultrasonic signals may be detected by the ultrasonic transducers. The ultrasonic signals may also be digitized and recorded by the integrated digitizers and the thermoacoustic data transmitted to the computing device.

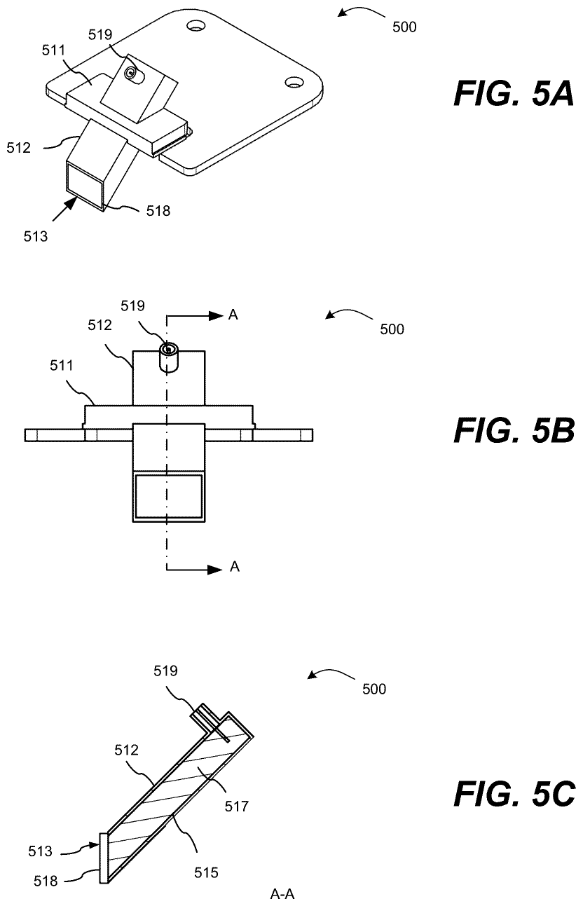

FIG. 5A is an isometric drawing depicting components of a TAT/UST system 500 with a single radio frequency (RF) antenna apparatus 512, according to embodiments. FIG. 5B is a front view drawing of components of TAT/UST system 500 in FIG. 5A. FIG. 5C is a cross-sectional drawing of components of TAT/UST system 500 in FIG. 5A.

RF antenna apparatus 512 is configured to couple radio frequency energy (e.g., pulses of RF waves) into a specimen being imaged. RF antenna apparatus 512, 514 includes an outer wall 515 that functions as an RF antenna and a distal end 513. RF antenna apparatus 512, 514 also includes a radio frequency connector 519 attached to outer wall 515. The radio frequency connector 519 is in electrical communication with at least one RF source. The pulses of radio frequency energy are sent from the at least one RF source to the RF antenna via the radio frequency connectors 519.

In the illustrated example, RF antenna apparatus 512 includes an inner volume 517 at least partially filled with dielectric material. The dielectric material may improve coupling of radio frequency energy into a specimen being imaged. In one aspect, the dielectric material has permittivity that is the same, or approximately the same (e.g., within 10%), as the permittivity of a specimen being imaged. For example, the dielectric material may have the same permittivity as a biological tissue. The dielectric material may improve coupling of radio frequency energy into a specimen being imaged. RF antenna apparatus 512 also includes a gas-filled spacer 518 (e.g., an air-filled spacer) at distal end 513. Gas-filled spacer 518 may block thermoacoustic signals generated inside the RF antenna apparatus. During operation, distal end 513 may be the side of RF antenna apparatus 512 facing the specimen to help prevent thermoacoustic signals generated inside RF antenna apparatus 512 from propagating into the specimen.

FIG. 6A is a drawing of components of an ultrasonic sensor array apparatus 630, according to an embodiment. FIG. 6B is an isometric drawing of components of the ultrasonic sensor array apparatus 630 in FIG. 6A.

Ultrasonic sensor array apparatus 630 includes an ultrasonic sensor array 650 having a ring frame 651 and plurality of ultrasonic transducers (e.g., 512 ultrasonic transducer elements), pre-amplifiers 770, low-pass filters 772, and digitizers 781. The ring frame 651 includes a ring-shaped printed circuit board (PCB). The integrated pre-amplifiers 670 are electrically connected to the ring-shaped PCB. The ultrasonic transducers are in electrical communication with the integrated pre-amplifiers 670. Integrated pre-amplifiers 670 may be used to increase the signal strength in the ultrasonic transducers. Each ultrasonic transducer is in electrical communication with one integrated pre-amplifier 670 to provide thermoacoustic signals and/or ultrasonic signals. Each integrated pre-amplifier 670 is in electrical communication with one low-pass filter 672 (e.g., low-pass filter with a cutoff frequency of about 2.2 MHz). Low-pass filters 672 may be used to avoid aliasing when sampling. Each low-pass filter 672 is in electrical communication with one digitizer 681 for parallel digitization. The integrated pre-amplifier 670 and ultrasonic transducer pairs are distributed along the circumference of the ring-shaped PCB.

Ultrasonic sensor array apparatus 630 also includes an ultrasonic transmitting transducer (transmitter) 633 having a diverging lens 634 with a field-of-view 635. Ultrasonic sensor array apparatus 630 also includes mechanical gear 652 and a motorized rotational shaft 637. The ultrasonic transmitting transducer (transmitter) 633 is coupled to the mechanical gear 652. The mechanical gear 652 is coupled to the motorized rotational shaft 637 to move/rotate (shown as arrow) the ultrasonic transmitting transducer (transmitter) 633 in a circular path around a specimen 636 during operation.

Ultrasonic sensor array apparatus 630 also includes conduits 638 within which wiring (e.g., one or more cables) passes between a matching network 682 and ultrasonic transmitting transducer 633 and/or between ultrasonic sensor array 650 to outside electrical components. The matching network 682 is in electrical communication with a power amplifier 684 and power amplifier 684 is in electrical communication with an arbitrary waveform generator (AWG) 686 that provides ultrasonic pulses. Matching network 682 may include a circuit of inductors and capacitors for increasing coupling to ultrasonic transmitting transducer (transmitter) 633. The AWG 686 can provide a custom voltage such as, e.g., a chirp signal sweeping over the bandwidth of the ultrasonic transducers (e.g., duration about 400 us long, and frequency about MHz). In one aspect, AWG 686 may be set to boost the SNR of the AST/UST system while maintaining safety standards for peak pressure used in ultrasound.

In some cases, the specimen 636 may be at least partially immersed in an acoustic medium during operation. Ultrasonic transmitting transducer (transmitter) 633 may be used to transmit ultrasonic signals through the acoustic medium into specimen 636. Transmitted, reflected, and/or scattered ultrasonic signals may then be detected by the ultrasonic transducers of ultrasonic sensor array 650 as ultrasonic transmitting transducer (transmitter) 633 is moved/rotated along the circular path.

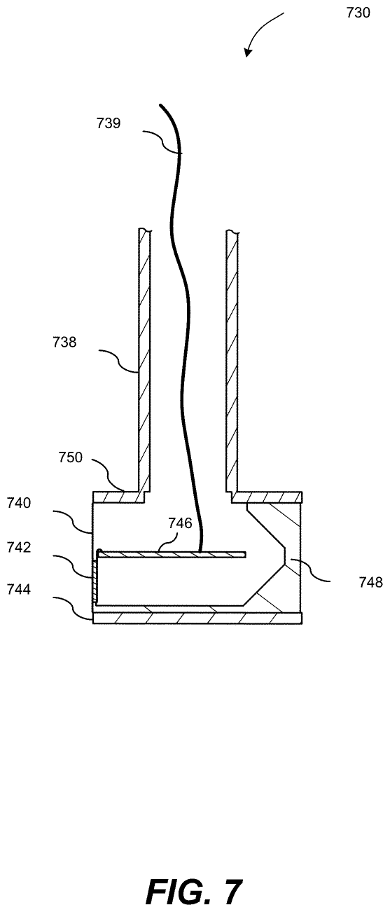

FIG. 7 is a cross-sectional drawing of components of an ultrasonic sensor array 750 of an ultrasonic sensor array apparatus 730, according to an embodiment. Ultrasonic sensor array 750 includes a printed circuit board (PCB) 746 having integrated pre-amplifiers, which are coupled in one-to-one correspondence with ultrasonic transducers. Components of an ultrasonic sensor array 750 are provided on a plastic support 744 (e.g., support made of polylactic acid plastic). Ultrasonic sensor array 750 also includes a front electrode 740 and a rear electrode 742 of each ultrasonic transducer. Ultrasonic sensor array 750 also includes stainless steel plates 748. The integrated pre-amplifiers 670 may be used to increase the signal strength in the ultrasonic transducers. Ultrasonic sensor array 750 also includes a conduit 738 within which wiring 739 (e.g., one or more cables) passes for connection to PCB 746.

FIG. 8A is an isometric drawing of components of a TAT/UST system 800 with a ring-shaped ultrasonic sensor array apparatus 830, according to embodiments. FIG. 8A is side view drawing of TAT/UST system 800 of FIG. 8A. FIG. 8C depicts a cross-sectional drawing of ring-shaped ultrasonic sensor array apparatus 830 of FIGS. 8A and 8B, according to embodiments. The illustrated example is shown during an operation in which a specimen 801 (e.g., human subject) is positioned within an inner diameter 802 of ring-shaped ultrasonic sensor array apparatus 830. The portion of the specimen 801 being imaged may be submerged in an acoustic medium (e.g., water solution) in e.g., a tank.

Ultrasonic sensor array apparatus 830 includes an ultrasonic sensor array 850 having a ring frame 851 and a plurality of ultrasonic transducers (e.g., 512 ultrasonic transducer elements). In one aspect, the ring frame 851 includes a ring-shaped printed circuit board (PCB) with integrated pre-amplifiers in one-to-one correspondence with the ultrasonic transducers. Ultrasonic sensor array apparatus 830 also includes an ultrasonic transmitting transducer (transmitter) 833 having a diverging lens 834. Ultrasonic sensor array apparatus 830 also includes mechanical gear 852 and a motorized rotational shaft 837. The ultrasonic transmitting transducer (transmitter) 833 is coupled to the mechanical gear 852, which is powered by a motor inside a housing box 858. The mechanical gear 852 is coupled to the motorized rotational shaft 837 to move/rotate (shown as double-sided arrow) the ultrasonic transmitting transducer (transmitter) 833 in a circular path during operation. Ultrasonic sensor array apparatus 630 also includes conduits 838 within which wiring (e.g., one or more cables) passes to ultrasonic sensor array 850 and/or to transmitting transducer 833.

During operation, ultrasonic transmitting transducer (transmitter) 833 may be used to transmit ultrasonic signals through the acoustic medium into specimen 801 during operation. Transmitted, reflected, and/or scattered ultrasonic signals may then be detected by the ultrasonic transducers of ultrasonic sensor array 850 as ultrasonic transmitter 833 is moved/rotated and transmits ultrasonic pulses. Ultrasonic transmitter 833 is rotated around specimen 801 using the mechanical gear 852 powered by the motor inside the housing box 858.

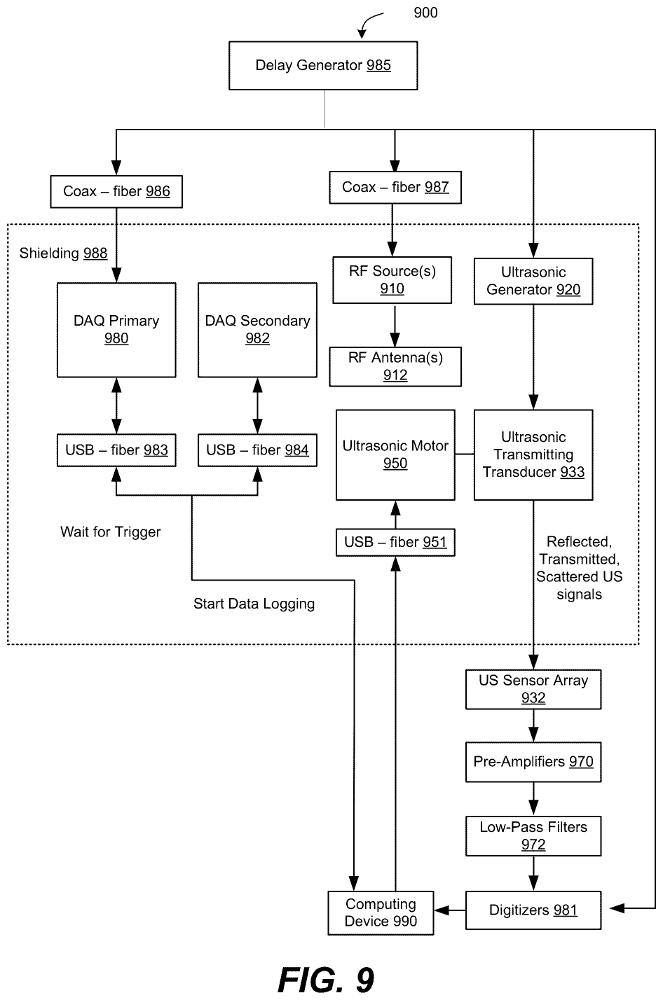

FIG. 9 is a block flow diagram showing control of radio frequency, ultrasonic, and digital components of a TAT/UST system 900, according to various embodiments. TAT/UST system 900 includes one or more radio frequency (RF) sources 910 (e.g., microwave source) and one or more radio frequency antennas 912 in communication with RF source(s) 910 to receive RF energy. During operation, radio frequency antenna(s) 912 may irradiate a specimen with ultrasonic pulses. TAT/UST system 900 also includes an ultrasonic pulser 920 for providing ultrasonic pulses and an ultrasonic transmitting transducer (ultrasonic transmitter) 933 for transmitting the ultrasonic pulses into the acoustic medium. TAT/UST system 900 also includes an ultrasonic motor 950 that is operably coupled to ultrasonic transmitting transducer 933 to move it along a path (e.g., a circular path around a specimen being imaged) while ultrasonic transmitting transducer 933 provides ultrasonic pulses.

TAT/UST system 900 also includes a DAQ primary 980, a DAQ secondary 982, a delay generator 985, and a computing device 990. The DAQ primary 980 triggers the DAQ secondary 982. Delay generator 985 is in electrical communication with one or more RF sources 910 via coax-fiber 987 and in electrical communication with DAQ primary 980 via coax-fiber 986. Computing device 990 is in electrical communication with DAQ primary 980 via universal serial bus (USB)-fiber 983, in electrical communication with DAQ secondary 982 via USB-fiber 984, and in electrical communication with ultrasonic motor 950 via USB-fiber 951. In other implementations, other electrical connectors may be employed.

TAT/UST system 900 also includes an ultrasonic sensor array 932 with a plurality of ultrasonic transducer elements, pre-amplifiers 970, low-pass filters 972, and digitizers 981. Pre-amplifiers 970 may be used to increase the signal strength from signals received from the ultrasonic transducers in ultrasonic sensor array 932. In one aspect, each ultrasonic transducer is in electrical communication with one integrated pre-amplifier 970 to provide thermoacoustic signals and/or ultrasonic signals. Each integrated-amplifier 670 is in electrical communication with one low-pass filter 972 (e.g., 2 MHz low pass filter). Low-pass filters 972 may be used to avoid aliasing when sampling. Each low-pass filter 972 may be in electrical communication with one digitizer 981 for parallel digitization implementation.

TAT/UST system 900 also includes an electromagnetic shielding 988 around certain components of TAT/UST system 900. Shielding 988 may shield waves generated by the certain components of TAT/UST system 900 from propagating to ultrasonic sensor array 932.

During operation, delay generator 985 alternates sending RF and ultrasonic signals. Delay generator 985 may include one or more processors and other circuitry and computer readable media. Delay generator 985 may include a timing circuit (e.g., an external timing circuit) which can alternate sending RF and ultrasonic signals.

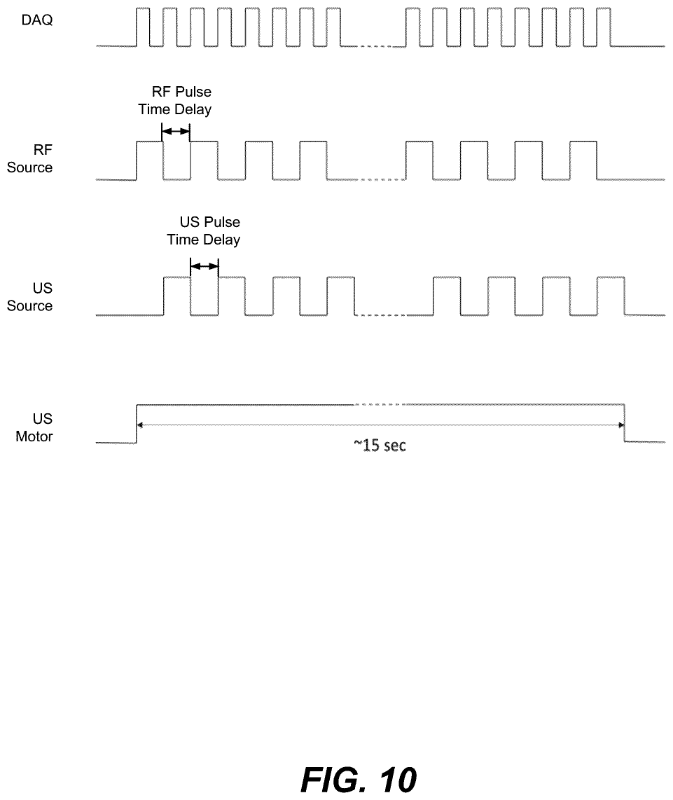

FIG. 10 is a timing diagram for alternating radio frequency and ultrasonic signals in a TAT/UST system, according to embodiments. During operation, a timing circuit alternates sending RF and ultrasonic signals. In one instance, a delay generator may include a time circuit that transmits digital trigger signals to the one or more radio frequency sources (e.g., radio frequency source(s) 110 of FIG. 1) and one or more ultrasonic sources (e.g., ultrasonic pulser 120 of FIG. 1) to control parallel digitization by digitizers (e.g., digitizers 181) of the recorded ultrasonic signals from an ultrasonic sensor array (e.g., ultrasonic sensor array 132 in FIG. 1). The delay generator may send trigger signals to RF source(s) and ultrasonic sources to generate time delays in the transmission of pulses of RF waves and signals of ultrasonic waves to interleave the RF pulses with the ultrasonic pulses. In the illustrated example, the delay generator sends trigger signals to generate a radio frequency (RF) pulse time delay between RF pulses and an ultrasonic (US) pulse time delay between ultrasonic pulses to generate an interleaved sequence of pulses while there is ongoing data acquisition by the ultrasonic sensors (transducers) of the ultrasonic sensor array. In this way, both thermoacoustic and ultrasonic TAT/UST data can be recorded during the same image data acquisition session. According to various embodiments, the imaging session may last in a range of seconds to minutes. In the illustrated example, the US motor moves the transmitter during a session lasting about 15 seconds.

### III. TAT/UST Methods

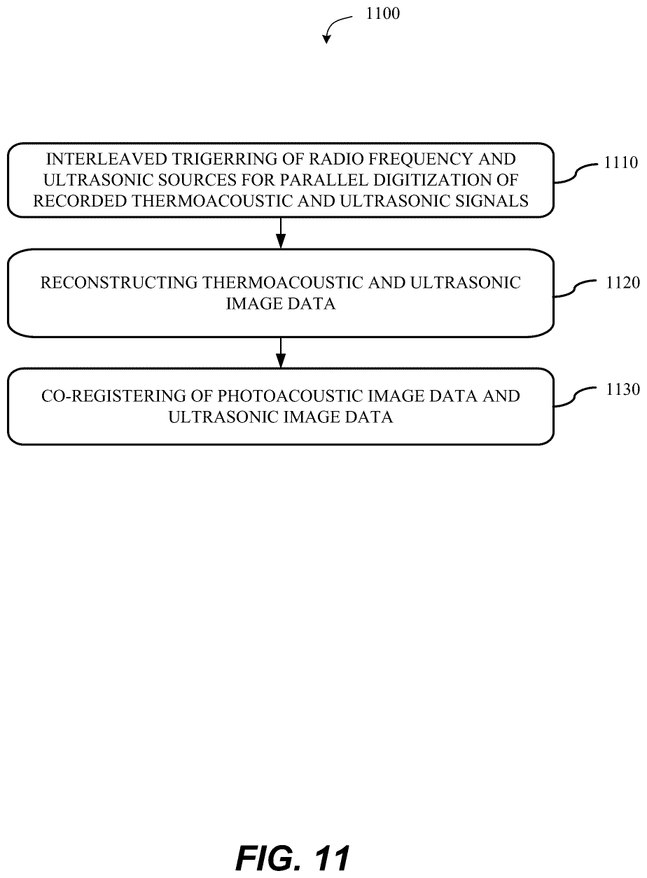

FIG. 11 depicts a flowchart 1100 of a TAT/UST method, according to embodiments. At operation 1110, control signals are sent to one or more components of a TAT/UST system to control triggering of radio frequency and ultrasonic sources for parallel digitization of thermoacoustic and ultrasonic signals. For example, a delay generator may be employed to send trigger signals to one or more radio frequency sources (e.g., radio frequency source(s) 110 of FIG. 1) and one or more ultrasonic sources (e.g., ultrasonic pulser 120 of FIG. 1) to generate time delays in the transmission of pulses of RF waves and signals of ultrasonic waves to interleave the RF pulses with the ultrasonic pulses while there is ongoing data acquisition by an ultrasonic sensor array. An example of a timing diagram is shown in FIG. 10. The pulses of RF waves cause absorption-specific thermoelastic expansion in the specimen which is released as thermoacoustic waves. The ultrasonic sensor array detects acoustic waves generated by the thermoelastic expansion and also detects ultrasonic waves transmitted, reflected, and/or scattered by the specimen.

At operation 1120, thermoacoustic and ultrasonic image data is reconstructed from the raw thermoacoustic and ultrasonic signals detected by the ultrasonic sensor array. Image reconstruction may include (i) reconstructing one or more two-dimensional images and/or (ii) reconstructing a volumetric three-dimensional image for a volume scanned by the ultrasonic sensor array. Image reconstruction includes, at least in part, implementing an inverse reconstruction algorithm. Some examples of inverse reconstruction methods that can be used include: (i) forward-model-based iterative methods, (ii) time-reversal methods, and (iii) back projection methods. A 3D back projection method can be used to reconstruct a 3D volumetric image and a 2D back projection method can be used to reconstruct a 2D image. An example of a back projection method is the universal back-projection process described in U.S. patent application Ser. No. 17/090,752, titled “SPATIOTEMPORAL ANTIALIASING IN PHOTOACOUSTIC COMPUTED TOMOGRAPHY” and filed on Nov. 5, 2020, which is hereby incorporated by reference for this description. Another example of a back-projection method can be found in Anastasio, M. A. et al., “Half-time image reconstruction in thermoacoustic tomography,” IEEE Trans., Med. Imaging 24, pp 199-210 (2005). In another aspect, a dual-speed-of sound (dual-SOS) photoacoustic reconstruction process may be used. An example of a single-impulse panoramic photoacoustic computed tomography system that employs a dual-SOS photoacoustic reconstruction process is described in U.S patent application 2019/0307334, titled “SINGLE-IMPULSE PANORAMIC PHOTOACOUSTIC COMPUTED TOMOGRAPHY” and filed on May 29, 2019.

At operation 1140, the reconstructed thermoacoustic and ultrasonic image data may be co-registered. The co-registered data may allow for anatomical structure from the ultrasonic image and contrast from the thermoacoustic image.

### IV. Examples of TAT/UST Techniques with Microwave Signals

In certain embodiments, TAT/UST systems include one or more RF antennas that are configured to irradiate with microwave pulses. These TAT/UST systems benefit from both rich microwave contrast and mm-scale fine acoustic resolution. In one aspect, to achieve a certain level of sensitivity, the one or more RF antennas are configured to provide high peak power (e.g., 10s-100s kW) but brief (e.g., <1 μs) microwave pulses that can radiate into a specimen, resulting in detectable pressures waves from thermoelastic expansion. For example, these TAT/UST systems can be used for human abdominal imaging.

According to one aspect, the RF antenna(s) are configured to provide brief pulses of microwaves (e.g., generally about 0.3-3 GHz) to illuminate a specimen, which leads to absorption-specific thermoelastic expansion released as thermoacoustic waves. Acoustic signals may then be detected (e.g., outside the body) by an ultrasonic sensor array and the acoustic signals can be used for image reconstruction of the absorption profile. The resulting acoustic images have contrast relating to water and ionic content and may provide mm-scale resolution of, e.g., biological structures within the body.

### A. Specific Absorption Rate (SAR)

According to one aspect, a TAT/UST system includes one or more RF sources and one or more RF antennas that are configured to provide pulsed RF waves (e.g., pulsed microwaves) with one or more parameters. The one or more parameters may be determined from a calculated specific absorption rate (SAR) at the surface of a specimen being imaged.

For example, when irradiated by pulsed microwaves with duration Δt (shorter than the thermal and stress confinement times for a characteristic length of interest as discussed in L. V. Wang and H. Wu, Biomedical optics: principles and imaging, John Wiley & Sons (2012)), the initial pressure rise p₀ at some region (position) r is found as:

p₀(r)=σₜ(r)|E(r)|₂ΓΔt=ρSAR(r)ΓΔt (Eqn. 1)

where σₜ is the local tissue conductivity (with both dielectric and ionic contributions), E is the local electric field amplitude, and Γ is the dimensionless Grüneisen parameter (about 0.2). The initial pressure can similarly be calculated from the specific absorption rate (SAR) and tissue density ρ (assumed 1000 kg·m₋₃). Non-magnetic tissues are generally assumed. The initial pressure rise then propagates as thermoacoustic waves which are detected outside the body using tens to thousands of acoustic sensors.

The image signal to noise ratio (SNR) is a key metric when designing and assessing TAT systems, and it motivates the use of high-power sources. SNR can be calculated by comparing the initial pressure rise in the tissue with the noise equivalent pressure (NEP) from the acoustic sensors projected into the tissue as discussed in A. M. Winkler, K. I. Maslov, and L. V. Wang, “Noise-equivalent sensitivity of photoacoustics,” J. Biomed. Opt., vol. 18, no. 9, p. 097003 (September 2013). For example, NEP of ultrasonic sensors in a sensor array is estimated to be Pa at the transducer surface over a 1 MHz bandwidth. To project into the imaging domain at distance r from an unfocused sensor, this value can be scaled by (r/λₐ) where λₐ is the acoustic wavelength. The acoustic wavelength is discussed in D. C. Garrett and L. V. Wang, “Acoustic sensing with light,” Nat. Photonics, vol. 15, no. 5, pp. 324-326 (2021). SNR at a desired imaging depth d inside the tissue is then found when averaged over the number of pulses N_{avg} and sensor elements Nₑₗₑ and by accounting for microwave attenuation. First, a plane wave model with uniform tissue properties with attenuation coefficient α according to following is used:

$$SNR ( d ) = {p}_{0} ( d ) \frac{\sqrt{{N}_{avg}} \sqrt{{N}_{ele}} {σ}_{t}}{NEP ( \frac{r}{{λ}_{a}} )} = ρ {SAR}_{0} {e}^{- 2 α d} ΓΔ t \frac{\sqrt{{N}_{avg}} \sqrt{{N}_{ele}} {σ}_{t}}{NEP ( \frac{r}{{λ}_{a}} )} \qquad ( Eqn . 2 )$$

For example, using a representative SNR of 10, N_{avg} of 1024, Nₑₗₑ of 512, r of 30 cm, λₐ of 1.5 mm, d of 5 cm, and α of 0.3 cm₋₁, this calculation yields an approximate required instantaneous SAR₀ at the tissue surface of 100 kW/kg.

This SAR calculation indicates a potential benefit of using high incident energy density onto the tissue. An implementation of a TAT/UST system according to the SAR calculation includes an RF source with a 400 kW, 1.1 GHz pulsed cavity magnetron which applies 0.2-1.0 μs pulses at fᵣₑₚ of 10-100 Hz. In one case, the RF source may be divided into multiple channels such as eight channels, each channel connected to a RF antenna such as a dielectric-loaded antenna. The dielectric-loaded antenna may be used to couple to couple microwave energy into human tissue, resulting in microwave absorption in a region of several kg.

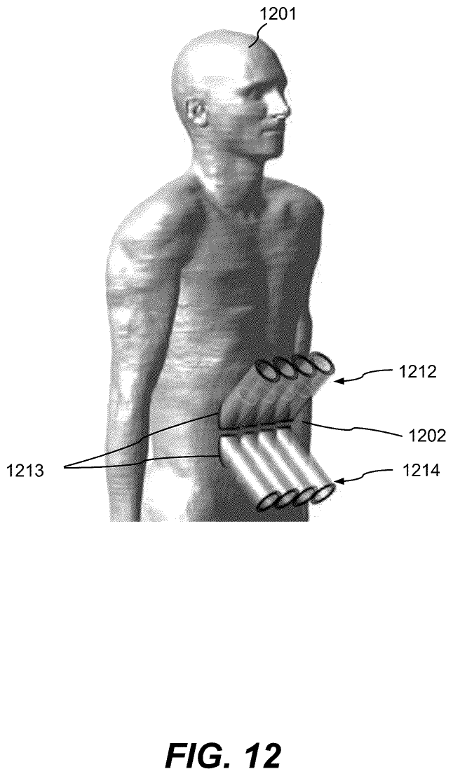

FIG. 12 is an illustration of a TAT/UST system with a radio frequency source (now shown) having a 400 kW, 1.1 GHz pulsed cavity magnetron which can apply 0.2-1.0 μs pulses at fᵣₑₚ of 10-100 Hz, according to an embodiment. The TAT/UST system includes eight dielectric-loaded RF antennas 1212, 1214 having distal ends 1213. In the illustrated example, distal ends 1213 are shown in contact with the skin of an abdomen 1202 of a human specimen 1201 during an imaging operation. Each of the RF antennas 1212, 1214 is configured to apply 50 kW peak power at 1.1 GHz. The RF source is divided into eight channels where each channel is connected to one of the eight dielectric-loaded RF antennas 1212, 1214.

### B. Microwave Safety Standards and Demonstrations

According to one aspect, a TAT/UST system includes an RF source and RF antennas that can provide microwave pulses based on an IEEE C95.1 standard of safety for microwave radiation. IEEE C95.1 standards can be found in “IEEE C95.1-2019 IEEE Standard for Safety Levels with Respect to Human Exposure to Electric, Magnetic, and Electromagnetic Fields, 0 Hz to 300 GHz,”<<https://standards.ieee.org/ieee/C95.1/4940/>> {accessed Feb. 25, 2022}. The IEEE C95.1 standard for microwave radiation specifies the limits for partial-body exposure in terms of SAR to prevent excessive heating in tissue. Three dosimetric reference limits are relevant to a TAT/UST system (found in Table 5 of C95.1) with averaging times t_{avg}:
(i) Whole-body SAR is limited to 0.4 W/kg, t_{avg}=30 min
(ii) Local SAR over any 10 g of tissue in the head or trunk is limited to 10 W/kg, t_{avg}=6 min.
(iii) Specific absorption (J/kg) over any 0.1 s period must not exceed 20% of the maximum local SAR (W/kg), t_{avg}=6 min

Assuming rectangular-modulated pulses, the time-averaged SAR is found from the temporal peak value by scaling by Δt and repetition rate fᵣₑₚ. It is assumed that a human subject is continually exposed for 6 minutes.

The TAT/UST system of this implementation was evaluated based on these safety standards using electromagnetic simulation tools with a finite difference time domain (FDTD) solver (Sim4Life, ZMT Zurich MedTech AG, Zurich, Switzerland). A model of an adult male (34 years old and mass of 70.2 kg) with segmented tissues and assigned electrical properties was used. The models of the RF antennas were filled with a tissue-matching dielectric material (εᵣ=25) and used to apply incident microwave signals. Circular waveguide sources applying 50 kW at 1.1 GHz in the TE D mode were assigned to each RF antenna. Simulations of the TAT/UST system yielded expected SAR at regions throughout the body. An example of a model of an adult male with segmented tissues can be found in M.-C. Gosselin et al., “Development of a new generation of high-resolution anatomical models for medical device evaluation: the Virtual Population 3.0,” Phys. Med. Biol., vol. 59, no. 18, p. 5287 (2014). An example of electrical properties of biological tissues can be found in P. A. Hasgall, E. Neufeld, M. C. Gosselin, A. Klingenbock, and N. Kuster, “IT′IS Database for thermal and electromagnetic parameters of biological tissues,” Version, vol. 4.1 (February 2022).

Assuming all incident energy is absorbed, the 400 kW RF source at Δt of 0.6 μs resulted in 0.24 J absorbed per pulse. The whole-body SAR was then found by scaling by fᵣₑₚ and the subject mass. For instance, the whole-body SAR for a 50 kg subject remains below 0.4 W/kg for fᵣₑₚ<80 Hz. For subjects of lower mass, the repetition rate must be reduced proportionally.

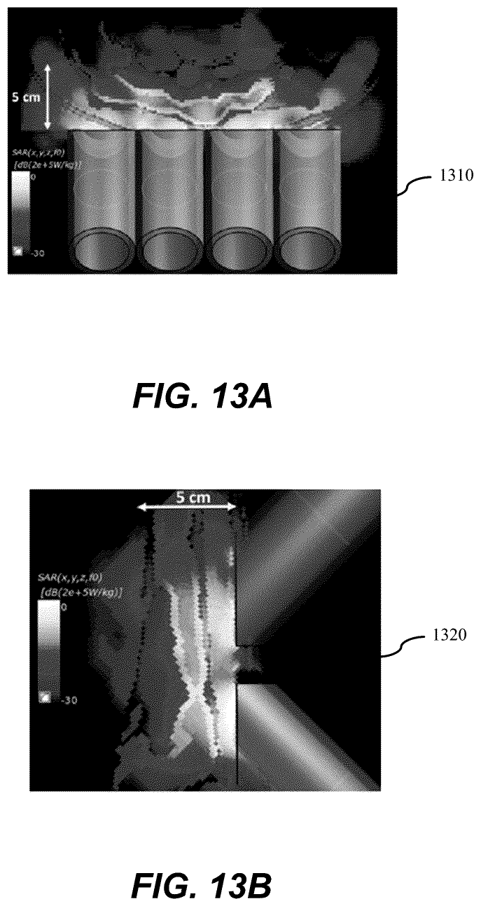

An example of a simulated instantaneous SAR distribution in the abdomen using the TAT/UST system of this implementation is shown in FIGS. 12A and 12B, in decibels with respect to 200 kW/kg. FIG. 13A is an image of a simulated SAR distribution into tissue of an abdomen of a human subject in the axial (left) plane using a TAT/UST system, in accordance with an implementation. FIG. 13B is an image of a simulated SAR distribution into tissue of an abdomen of a human subject in the sagittal (right) plane using a TAT/UST system, in accordance with an implementation.

Sim4Life may be used to assess tissue exposure according to the IEEE C95.3-2002 standard, used to evaluate C95.1 compliance. An example of an IEEE C95.3-2002 standard can be found at page 3 of “IEEE Recommended Practice for Measurements and Computations of Electric, Magnetic, and Electromagnetic Fields with Respect to Human Exposure to Such Fields, Hz to 300 GHz,” IEEE Std C953-2021 Revis. IEEE Std C953-2002 IEEE Std C9531-2010, pp. 1-240 (May 2021). The average SAR over any 10 g of tissue was evaluated, finding a maximum value of 208 kW/kg which is similar to our calculated requirement. For a pulse width of 0.6 μs, the time-averaged local SAR is then estimated as 0.125 [J·kg⁻¹]×fᵣₑₚ. Therefore, to maintain the localized SAR below 10 W/kg, fᵣₑₚ can similarly not exceed 80 Hz.

Based on these findings, using a maximum repetition rate in our system of 70 Hz therefore allows a ˜10% error margin in whole-body and localized SAR for subjects greater than 50 kg. Given a repetition rate of 10-70 Hz, if whole-body and local criteria are met, condition (iii) is also met. That is, over any 0.1 s period, the energy deposition is less than 20% of the time averaged value. The peak electric field in the RF antenna was found to be approximately 90 kV/m when transmitting 50 kW.

Some examples of guideline limitations and system level parameters for different SAR conditions are summarized in table below:

| | Guideline | System level |
|---|---|---|
| Condition | limitation | (at fᵣₑₚ = 70 Hz |
| (i) Whole- | 0.4 W/kg | 0.34 W/kg for a |
| body SAR | | 50 kg subject |
| (ii) Localized | 10 W/kg | 8.74 W/kg |
| SAR | | |
| (iii) Pulsed | 2 W/kg averaged | 0.87 W/kg |
| SAR | over 0.1 s | |
| (ii) Peak | (N/A, previously | 90 kV/m |
| electric field | 100 kV/m) | |

Modifications, additions, or omissions may be made to any of the above-described implementations without departing from the scope of the disclosure. Any of the implementations described above may include more, fewer, or other features without departing from the scope of the disclosure. Additionally, the steps of described features may be performed in any suitable order without departing from the scope of the disclosure. Also, one or more features from any implementation may be combined with one or more features of any other implementation without departing from the scope of the disclosure. The components of any implementation may be integrated or separated according to particular needs without departing from the scope of the disclosure.

It should be understood that certain aspects described above can be implemented in the form of logic using computer software in a modular or integrated manner. Based on the disclosure and teachings provided herein, a person of ordinary skill in the art will know and appreciate other ways and/or methods to implement the present invention using hardware and a combination of hardware and software.

Any of the software components or functions described in this application, may be implemented as software code using any suitable computer language and/or computational software such as, for example, Java, C, C#, C++ or Python, LabVIEW, Mathematica, or other suitable language/computational software, including low level code, including code written for field programmable gate arrays, for example in VHDL. The code may include software libraries for functions like data acquisition and control, motion control, image acquisition and display, etc. Some or all of the code may also run on a personal computer, single board computer, embedded controller, microcontroller, digital signal processor, field programmable gate array and/or any combination thereof or any similar computation device and/or logic device(s). The software code may be stored as a series of instructions, or commands on a CRM such as a random access memory (RAM), a read only memory (ROM), a magnetic medium such as a hard-drive or a floppy disk, or an optical medium such as a CD-ROM, or solid stage storage such as a solid state hard drive or removable flash memory device or any suitable storage device. Any such CRM may reside on or within a single computational apparatus, and may be present on or within different computational apparatuses within a system or network. Although the foregoing disclosed implementations have been described in some detail to facilitate understanding, the described implementations are to be considered illustrative and not limiting. It will be apparent to one of ordinary skill in the art that certain changes and modifications can be practiced within the scope of the appended claims.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain implementations herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or implementations of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

What is claimed is:

1. An imaging system, comprising:
one or more dielectric-loaded radio frequency antenna apparatus, each dielectric-loaded radio frequency antenna apparatus comprising a radio frequency antenna for transmitting radio frequency pulses and an internal volume at least partially filled with a dielectric material; and
an ultrasonic sensor array configured to detect acoustic waves.

2. The imaging system of claim 1, wherein the dielectric material has a permittivity property that approximates a permittivity property of a specimen being imaged during operation.

3. The imaging system of claim 1, further comprising a gas-filled spacer configured to block acoustic waves.

4. The imaging system of claim 3, wherein the gas-filled spacer is located between the one or more radio frequency antennas and an acoustic medium.

5. The imaging system of claim 1, further comprising an ultrasonic transmitting transducer.

6. The imaging system of claim 5, further comprising a delay generator configured to interleave thermoacoustic signals based on radio frequency energy from the one or more dielectric-loaded radio frequency antenna apparatus with ultrasound signals based on ultrasonic energy from the ultrasonic transmitting transducer.

7. The imaging system of claim 6, further comprising a data acquisition system configured to record and digitize the interleaved thermoacoustic and ultrasonic signals in a single recording session.

8. The imaging system of claim 5, wherein the ultrasonic transmitting transducer is configured to move around a specimen being imaged.

9. The imaging system of claim 5, further comprising a motor configured to move the ultrasonic transmitting transducer along a line, an arc, or a circle.

10. The imaging system of claim 5, wherein the ultrasonic transmitting transducer comprises a diverging lens.

11. The imaging system of claim 1, wherein the imaging system is configured for human-scale imaging.

12. The imaging system of claim 11, further comprising a tank with an acoustic medium.

13. The imaging system of claim 1, wherein the ultrasonic sensor array comprises a plurality of integrated pre-amplifiers in one-to-one correspondence with a plurality of ultrasonic transducers.

14. The imaging system of claim 13, wherein the ultrasonic transducers and pre-amplifiers are located along a ring.

15. The imaging system of claim 1, wherein the ultrasonic sensor array comprises a shield.

16. An ultrasonic sensor array apparatus, comprising:
a plurality of ultrasonic transducers;
a plurality of integrated pre-amplifiers in one-to-one correspondence with the ultrasonic transducers; and
an ultrasonic transmitter configured to transmit ultrasound pulses.

17. The ultrasonic sensor array of claim 16, further comprising a shield.

18. The ultrasonic sensor array of claim 16, further comprising a motor configured to move the ultrasonic transmitting transducer along a line, an arc, or a circle.

19. The ultrasonic sensor array of claim 18, further comprising a rotational shaft coupled to the motor and a gear coupled to the rotational shaft and wherein the ultrasonic transmitting transducer is coupled to the gear.

20. The ultrasonic sensor array of claim 16, wherein the ultrasonic transmitter comprises a diverging lens.

21. A dielectric-loaded radio frequency antenna apparatus, comprising:
an internal volume at least partially filled with a dielectric material; and
a radio frequency antenna;
a gas-filled spacer between the internal volume and a region external to the dielectric-loaded radio frequency antenna.

22. The dielectric-loaded radio frequency antenna of claim 21, wherein the dielectric material has a permittivity property that approximates a permittivity property of a specimen being imaged during operation.

23. The dielectric-loaded radio frequency antenna of claim 21, wherein the radio frequency antenna is configured to generate microwave pulses.

24. The dielectric-loaded radio frequency antenna of claim 21, further comprising a distal end configured to contact a specimen being imaged.

25. A thermoacoustic and ultrasound tomography imaging method:
delayed triggering of radio frequency and ultrasonic pulses to interleave thermoacoustic signals with ultrasonic signals detected by an ultrasonic sensor array;
reconstructing thermoacoustic image data and ultrasonic image data; and
co-registering the thermoacoustic image data and ultrasonic image data.
